(19) United States
(12) Patent Application Publication
(10) Pub. No.: US 2003/0195583 A1
(43) Pub. Date: Oct. 16, 2003

(54) Method of relieving anxiety or promoting relaxation

(75) Inventors: Gregory J. Gruzdowich, Carlsbad, CA (US); Thomas L. Grey, Carlsbad, CA (US)

Correspondence Address: CROCKETT & CROCKETT, 24012 CALLE DE LA PLATA, SUITE 400, LAGUNA HILLS, CA, 92653, US

(73) Assignee: Woodside Biomedical, Inc.

(21) Appl. No.: 10/421,349

(22) Filed: Apr. 22, 2003

Related U.S. Application Data
(63) Continuation of application No. 09/444,581, filed on Nov. 18, 1999.

(51) Int. Cl. A61N 1/18

(52) U.S. Cl. 607/45

(57) ABSTRACT

A device for providing noninvasive electrical stimulation of a single acupuncture site for treatment of anxiety is disclosed.

This application is a continuation application of U.S. application Ser. No. 09/444,581, filed Nov. 18, 1999.

### FIELD OF THE INVENTIONS

The methods and devices described below relate to the fields of treatment of anxiety and noninvasive electrical stimulation of an acupuncture point.

### BACKGROUND OF THE INVENTIONS

Acupuncture has been proposed for the treatment of anxiety. Garcia-Rill, Surface Stimulation of Specific Acupuncture Points to Reduce Anxiety, U.S. Pat. No. 5,950,635 (Sep. 14, 1999) proposes applying electrical stimulation to the acupuncture points LR3, HT3 and PC6 to relieve anxiety. In accordance with well-known acupuncture standards, several acupuncture points are simultaneously stimulated to achieve the therapeutic goal. The several points are widely distributed on the body, with one point on the wrist (PC6), one point on the foot (LR3), and one point on the elbow (HT3).

Bertolucci, Nausea Control Device, U.S. Pat. No. 4,981,146, Jan. 1, 1991, describes a nausea control device in the form of a watch-like housing attachable to the human wrist by an adjustable attachment band. The device uses non-invasive nerve stimulation whereby electricity is passed through two electrodes to stimulate nerves located on the ventral side of the wrist (this anatomical position is sometimes referred to as the palmar side of the wrist. The treatment provided by the device is sometimes referred to as electro-acupuncture which is a form of acupuncture, and the ventral site of application is referred to in the acupuncture art as the P6 point, pericardium 6 point, or master point of the pericardium meridian (sometimes referred to as the vascular meridian). A primary object of the invention is to provide a non-chemical, non-invasive, painless and inexpensive method of alleviating nausea. It is also portable, self-contained and convenient to the patient. Electrical pulse repetition rate of approximately 70 pulses per second and a pulse width of 80 microseconds has been found to provide effective relief of nausea in a patient. Our currently preferred electrical pulse pattern comprises about 350 microsecond pulse width at about 31 pulses per second at power levels of about 10-35 milli-amps peak pulse height. Thus a wide range of pulse patterns may be used in non-invasive nerve stimulation devices.

We have discovered that using noninvasive electrical stimulation of the P6 or Neiguan point of the pericardium meridian relieves anxiety and phobic reactions. The effect is obtained without stimulation of other acupuncture points. It takes only a few minutes of stimulation to achieve marked reduction in anxiety and phobic reactions.

### SUMMARY

The method described below employs use of the device described in Bertolucci, Nausea Control Device, U.S. Pat. No. 4,981,146 (Jan. 1, 1991) and similar devices for the relief and alleviation of anxiety.

### BRIEF DESCRIPTION OF THE DRAWINGS

FIG. 1 illustrates placement of an electro-acupuncture device over the P6 acupuncture point on the human wrist.

FIG. 2 illustrates a stimulation waveform for stimulating the wrist in accomplishing the treatment.

FIG. 3 illustrates an individual pulse of the stimulation waveform.

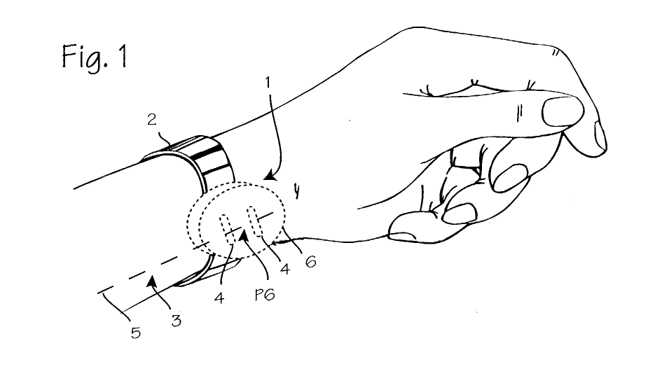

### DETAILED DESCRIPTION OF THE INVENTIONS

Use of our ReliefBand® NST™ device for the approved treatment of nausea has revealed that the treatment also relieves anxiety. Significant reduction in severe anxiety and/or phobia, such as severe driving anxiety, has been observed. Anecdotal reports indicate that the electrostimulation provided to the P6 point on the wrist alleviates performance anxiety such as that experienced while golfing, and encourages relaxation. The ReliefBand® NST™ is a wristwatch like device worn on the wrist and energized to provide electrical stimulation to the wrists. The ReliefBand® NST™ non-invasive nerve stimulation device 1 is secured with strap 2 to the ventral side of the wrist 3 such that the pair of electrodes 4 are disposed over the median nerve 5 (indicated by the phantom line) in contact with the skin in the vicinity of the P6 acupuncture point. The electrodes are on the underside of the housing 6, the required battery and control electronics are housed within the housing, and input mechanisms are located on the outer face of the housing. The electrodes stimulate the median nerve and collateral or associated nerve structures.

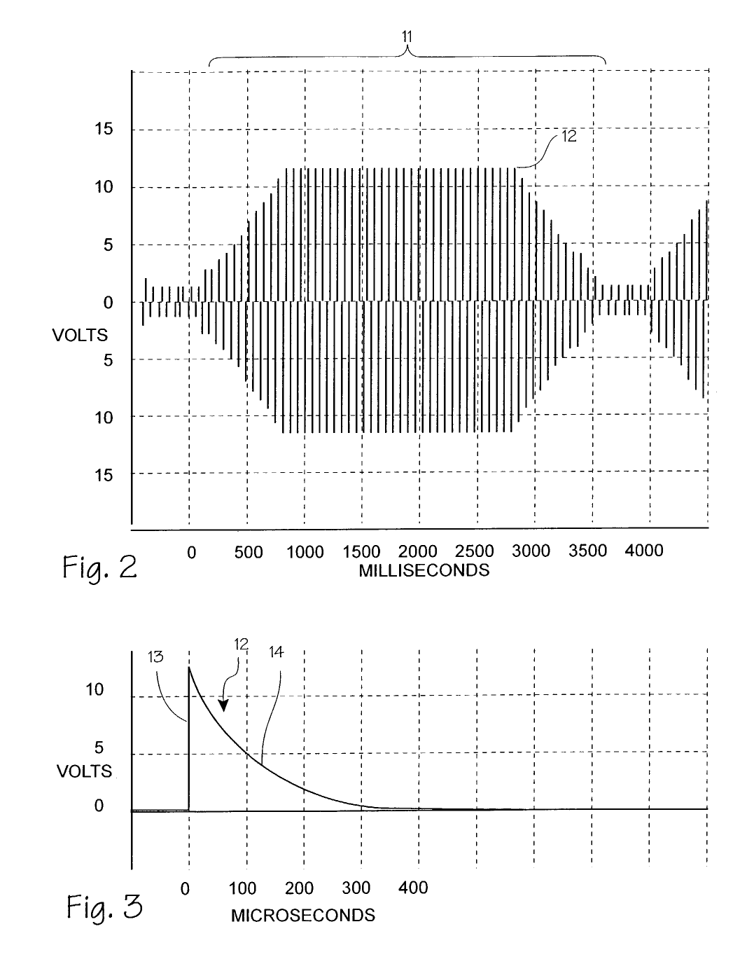

FIG. 2 shows the preferred waveform. The overall waveform comprises a series of bipolar trapezoidal waveform which make low frequency pulses 11. The waveform is initiated at low power levels of about 1 to 2 volts and ramps up over a period of about 1 second to a maximum level of 11-12 volts, and is maintained for about 2 seconds, and then ramps down over a period of about 1 second to low power levels of about 1 to 2 volts. The individual pulses 12 are separated by about 32 milliseconds (msec)(measured peak to peak), and last about 350 microseconds (μsec). The individual pulses alternate between negative and positive pulses, and are said to constitute a bipolar waveform. The individual pulses are illustrated in FIG. 3, in which the time scale is enlarged to show the detail. The individual pulse 12 is made of a sharply vertical spike which decays exponentially over a period of about 350 μsec, thus comprising a basically vertical leading edge 13 and an exponentially decaying trailing edge 14 to each individual pulse. The following pulse will be shaped the same, except that it will be of negative voltage. The exponential nature of the individual pulse decay maximizes the high frequency components in the signal. These high frequency components contribute to a lessening of the skin impedance, in particular the capacitive components. This contributes to a higher level of current able to enter the deeper tissues. The power levels may be adjusted up or down to intensify the therapeutic effect of the device or lessen the sensation causes by the device, according to the preferences of individual users. The pulse rate within the waveform may be increased or decreased also.

To use the device to alleviate anxiety, the user merely secures the housing over the inner surface of the wrist and straps it on like a wristwatch. This places the electrodes over the P6 acupuncture point, in electrical contact with the skin overlying the median nerve. The user then turns the device on, adjusts it to a comfortable power level, and allows stimulation to continue for a few minutes, for example 5-10 minute to achieve relief. The device may be applied intermittently, once every hour or so, or continuously. The device provides electrical current and voltage to the electrodes for driving anxiety the device may be used for the entire time the user is driving. For phobias such as agoraphobia or fear of heights, the device may be used while the patient is situated in the environment causing the phobic reaction. For relief of sports anxiety, or to encourage relaxation during sporting events, the device may be used by the patient during the entire time the patient is participating in the event, such as during an entire round of golf or an entire baseball game, or it may be used intermittently during periods which the user is particularly susceptible to anxiety and its ill-effects on athletic performance. For promoting relaxation generally, or relieving non-specific anxiety, the device may be used for short periods of, for example, 10-20 minutes on an occasional basis. The device will eliminate the anxiety or phobic reactions entirely, or reduce them to tolerable levels. In addition to driving phobias, other phobic reactions such as flying phobia, fear of heights, etc. should be treatable in the same manner. While less convenient, the methods may be accomplished with electro-acupuncture needles or electrodes handled individually by an acupuncturist.

While the devices and methods have been described in reference to the environment in which they were developed, they are merely illustrative of the principles of the inventions. Other embodiments and configurations may be devised without departing from the spirit of the inventions and the scope of the appended claims.

We claim:

1. A method of relieving anxiety in a patient comprising the steps of:
mounting a non-invasive nerve stimulation device onto the ventral side of the wrist;
generating a stimulation signal; and
delivering the stimulation signal to the ventral side of the wrist;
leaving acupuncture points located remotely from the wrist untreated by the delivery of a stimulation signal.

2. A method of relieving anxiety in a patient comprising the steps of:
mounting at least one electrode onto the ventral side of the wrist;
generating a stimulation signal; and
delivering the stimulation signal to said at least one electrode to stimulate the ventral side of the wrist;
leaving acupuncture points located remotely from the wrist untreated by the delivery of a stimulation signal.

3. The method of claim 2 wherein said mounting step includes providing a wristwatch-like housing carrying the at least one electrode, and providing securing means for mounting the housing onto the wrist, with the housing having a circuit means for generating the stimulation signal encased within the housing.

4. The method of claim 2 wherein said delivering step comprises delivering an intermittent stimulation signal.

5. The method of claim 2 wherein said delivering step comprises delivering a continuous stimulation signal.

6. A method of relieving anxiety in a patient by stimulating the median nerve and associated nerve structures in the wrist of the patient with electrical energy without stimulating nerves located remotely from the wrist of the patient.

7. A method of relieving anxiety in a patient, said method comprising the steps of:
applying an electro-acupuncture device onto the wrist;
stimulating the wrist with the acupuncture, acupressure or electro-acupuncture device;
wherein said stimulating the wrist is not accompanied by stimulation of the LR3 and HT3 acupuncture points.

8. A method of improving athletic performance, said method comprising:
applying an electro-acupuncture device onto the wrist;
stimulating the wrist with the electro-acupuncture device;
participating in an athletic activity;
wherein the step of stimulating the wrist is not accompanied by stimulation of the LR3 and HT3 acupuncture points.

9. A method of relieving driving anxiety suffered by a patient, said method comprising:
applying an electro-acupuncture device onto the wrist;
stimulating the wrist with the acupuncture, acupressure or electro-acupuncture device;
wherein the step of stimulating the wrist is not accompanied by stimulation of the LR3 and HT3 acupuncture points.

10. The method of claim 10 wherein the step of stimulating the wrist is performed while the patient is driving a moving vehicle.

11. The method of claim 10 wherein the step of stimulating the wrist is performed while the patient is in a moving vehicle.

12. A method for treating a patient suffering from an anxiety related disorder comprising the steps of:
engaging an electrode with the P6 acupuncture point on the patient
applying an electrical current to said electrode;
wherein the step of applying an electrical current to said electrode is not accompanied by stimulation of the LR3 and HT3 acupuncture points.
